(19) United States
(12) Patent Application Publication
(10) Pub. No.: US 2010/0322873 A1
(43) Pub. Date: Dec. 23, 2010

(54) BEVERAGE COMPRISING ARGININE

(75) Inventors: Rajnish Kohli, Hillsborough, NJ (US); Richard Scott Robinson, Belle Mead, NJ (US); Diane Cummins, Livingston, NJ (US); Nagaraja Jayaraman, Cos Cob, CT (US)

Correspondence Address: COLGATE-PALMOLIVE COMPANY, 909 RIVER ROAD, PISCATAWAY, NJ, 08855, US

(73) Assignee: COLGATE-PALMOLIVE COMPANY, New York, NY (US)

(21) Appl. No.: 12/866,760

(22) PCT Filed: Feb. 6, 2009
(86) PCT No.: PCT/US09/33307

§ 371 Date: Aug. 9, 2010

Related U.S. Application Data
(60) Provisional application No. 61/027,434, filed on Feb. 8, 2008.

(51) Int. Cl. A61K 8/18; A23L 2/00; A23L 2/02; A23L 2/52; A61Q 11/00

(52) U.S. Cl. 424/49; 426/590; 426/599; 426/72

(57) ABSTRACT

The invention provides a beverage comprising a basic amino acid and a calcium salt.

This application claims the benefit of U.S. Ser. No. 61/027,434 filed Feb. 8, 2008, the contents of which are incorporated herein by reference.

### FIELD OF THE INVENTION

The invention relates to beverages containing arginine, together with calcium, phosphate, and optionally other ions to promote oral health.

### BACKGROUND

Soft drinks containing high levels of sugar are extremely damaging to the teeth. Even diet sodas are damaging due to their high acidity. Sport drinks containing ions to replenish the body following exercise are popular, but tend to be highly sugared, again promoting tooth decay. There is a need for healthy, refreshing drinks which promote rather than damage oral health.

### SUMMARY OF THE INVENTION

Without intending to be bound by a particular theory, it is hypothesized that a significant factor in the beneficial effect of arginine is that arginine and other basic amino acids can be metabolized by certain types of bacteria, e.g., S. sanguis which are not cariogenic and which compete with cariogenic bacteria such as S. mutans, for position on the teeth and in the oral cavity. The arginolytic bacteria can use arginine and other basic amino acids to produce ammonia, thereby raising the pH of their environment, while cariogenic bacteria metabolize sugar to produce lactic acid, which tends to lower the plaque pH and demineralize the teeth, ultimately leading to cavities. Regular use of oral care products comprising arginine, over time, will lead to a relative increase in the arginolytic bacteria and a relative decrease in the cariogenic bacteria, resulting in a higher plaque pH, in effect immunizing the teeth against cariogenic bacteria and their detrimental effects. This effect is further enhanced by providing minerals such as calcium, and optionally other minerals such as phosphate and fluoride which promote remineralization of the teeth.

L-arginine and arginine salts such as arginine-bicarbonate, however, are by themselves distinctly bitter in taste and in solution can also impart a fishy taste. When these ingredients are incorporated in products at effective concentrations to impart anticavity efficacy and sensitivity relief, these ingredients might negatively impact the taste and mouthfeel of the finished product, particularly when directly compared to a matching formulation without the arginine free base or salt. Surprisingly, the addition of these ingredients results in a significant enhancement of taste and mouthfeel attributes, as well as increase in an overall acceptance of the products.

The invention thus provides a beverage (Formulation 1.0) comprising an effective amount of
a. a basic amino acid, e.g., arginine, in free or salt form, e.g., present in an amount of at least 0.1% (by weight of free base),
b. a calcium salt.
   For example, the invention provides

1.1. Formulation 1.0 further comprising a phosphate ion source, e.g., a soluble phosphate salt, e.g., potassium phosphate monobasic or dibasic potassium phosphate.

1.2. Formulation 1.0 or 1.1 further comprising a potassium ion source, e.g., potassium chloride or potassium phosphate monobasic or dibasic potassium phosphate.

1.3. Formulation 1.0, 1.1 or 1.2 further comprising a fluoride source, e.g., a soluble fluoride salt, e.g., sodium fluoride.

1.4. Any of the preceding formulations comprising a polyol, e.g., selected from glycerol, sugar alcohols (e.g., sorbitol, xylitol) and combinations thereof.

1.5. Any of the preceding formulations comprising xylitol.

1.6. Any of the preceding formulations wherein the calcium salt is selected from calcium carbonate, calcium hydroxide, calcium citrate, calcium malate, calcium lactate, calcium chloride, calcium glycerophosphate, calcium formate, and mixtures thereof.

1.7. Any of the preceding formulations comprising arginine bicarbonate.

1.8. Any of the preceding formulations comprising arginine phosphate.

1.9. Any of the preceding formulations comprising an organic acid, e.g., citric acid, malic acid or acetic acid.

1.10. Any of the preceding formulations which is carbonated.

1.11. Any of the preceding formulations further comprising fruit juice, fruit extract or fruit concentrate.

1.12. Any of the preceding formulations further comprising vitamins (e.g., water soluble vitamins, e.g., B-complex vitamins or ascorbic acid), minerals, antioxidants, and or preservatives.

1.13. Any of the preceding formulations further comprising herbal extracts, e.g., ginseng, green tea, black tea, ginko, guarana, and hoodia.

The beverages of the invention promote oral and systemic health in variety of ways, for example, the invention provides method to improve oral health comprising use of the beverages of formulations 1.0-1.13, e.g., by a subject in need thereof, to

a. reduce or inhibit formation of dental caries,

b. reduce, repair or inhibit early enamel lesions,

c. reduce or inhibit demineralization and promote remineralization of the teeth,

d. reduce hypersensitivity of the teeth,

e. reduce or inhibit gingivitis,

f. promote healing of sores or cuts in the mouth,

g. reduce levels of acid producing bacteria,

h. to increase relative levels of arginolytic bacteria,

i. inhibit microbial biofilm formation in the oral cavity,

j. raise and/or maintain plaque pH at levels of at least pH 5.5 following sugar challenge,

k. reduce plaque accumulation,

l. treat dry mouth,

m. whiten teeth,

n. enhance systemic health, including cardiovascular health, e.g., by reducing potential for systemic infection via the oral tissues,

o. reduce erosion of the teeth,

p. immunize the teeth against cariogenic bacteria, and/or

q. clean the teeth and oral cavity.

### DETAILED DESCRIPTION OF THE INVENTION

The basic amino acids which can be used in the compositions and methods of the invention include not only naturally occurring basic amino acids, such as arginine, lysine, and histidine, but also any basic amino acids having a carboxyl group and an amino group in the molecule, which are water-soluble and provide an aqueous solution with a pH of 7 or greater.

Accordingly, basic amino acids include, but are not limited to, arginine, lysin. citrullene, ornithine, creatine, histidine, diaminobutanoic acid, diaminoproprionic acid, salts thereof or combinations thereof in a particular embodiment, the basic amino acids are selected from arginine, citrullene, and ornithine.

In certain embodiments, the basic amino acid is arginine, for example, l-arginine, or a salt thereof.

The compositions of the invention are intended for consumption and so salts for use in the present invention should be safe for such use, in the amounts and concentrations provided. Suitable salts include salts known in the art to be pharmaceutically acceptable salts are generally considered to be physiologically acceptable in the amounts and concentrations provided. Physiologically acceptable salts include those derived from pharmaceutically acceptable inorganic or organic acids or bases, for example acid addition salts formed by acids which form a physiological acceptable anion, e.g., hydrochloride or bromide salt, and base addition salts formed by bases which form a physiologically acceptable cation, for example those derived from alkali metals such as potassium and sodium or alkaline earth metals such as calcium and magnesium. Physiologically acceptable salts may be obtained using standard procedures known in the art, for example, by reacting a sufficiently basic compound such as an amine with a suitable acid affording a physiologically acceptable anion.

The absolute concentration of calcium used in the compositions of the present invention is not critical as this will vary according to the nature and concentration of the acids present. The acid composition may contain organic and/or inorganic acids and may be supplemented with vitamins such as ascorbic acid. The calcium concentration may vary from 0.001 mol. per liter to more than 0.25 mol. per liter, typically from 0.002 mol. per liter to 0.1 mol. per liter, suitably from 0.01 mol. per liter to 0.05 mol. per liter. The calcium may be added in any suitable form, conveniently as a soluble salt such as calcium carbonate, calcium hydroxide, calcium citrate, calcium malate, calcium lactate, calcium chloride, calcium acetate, calcium glycerophosphate or calcium formate or arty other salt which minimizes any adverse flavor contribution to the composition.

Compositions of the invention may be prepared by (i) combining the arginine or basic amino acid with an acid in a Premix 1; (i) mixing an organic acid (e.g., citric acid) with its corresponding calcium salt (e.g., calcium citrate) or another calcium salt to form Premix 2; adding the other ingredients, and combining the Premixes. It may be advantageous to mix the acid with an alkaline calcium salt such as calcium carbonate or calcium hydroxide thereby minimizing the concentration of acid applied to the formulation. The acid can also be mixed with inorganic calcium salts such as calcium chloride. The molar ratio of calcium to acid is 0.3-0.55 or 0.4 to 0.55. Most preferably the molar ratio is at least 0.4, and a value of about 0.5 has been found to be especially effective.

Representative fluoride ion sources include, but are not limited to, stannous fluoride, sodium fluoride, potassium fluoride, sodium monafluorophosphate, sodium fluorosilicate, ammonium fluorosilicate, amine fluoride, ammonium fluoride, and combinations thereof. In certain embodiments the fluoride ion source includes stannous fluoride, sodium fluoride, sodium monofluorophosphate as well as mixtures thereof. In a particular embodiment, the fluoride source is sodium fluoride or sodium monofluorophosphate. Concentrations are generally less than 100 ppm, e.g., 1-25 ppm.

The products may be unsweetened or sweetened with sugars, sugar alcohols, or intense sweeteners such as saccharine, aspartyl phenyl, alanyl methyl ester, or other sweeteners known in the art.

In a particular embodiment, the product comprises xylitol, which has a sweet taste and is also antibacterial.

The products may also contain other conventional additives such as sodium benzoate, sorbic acid, sodium metabisulfite, ascorbic acid, flavorings and colorings.

The following examples further describe and demonstrate illustrate embodiments within the scope of the present invention. The examples are given solely for illustration and are not to be construed as limitations of this invention as many variations are possible without departing from the spirit and scope thereof. Various modifications of the invention in addition to those shown and described herein. should he apparent to those skilled in the art and are intended to fail within the appended claims.

### EXAMPLE 1

### Formulation Comprising Arginine

Formulations of the invention are prepared from the following ingredients:

Formula A

| RAW MATERIAL | WEIGHT % |
|---|---|
| Deionized Water | 96.26815 |
| Xylitol | 2.00000 |
| L-Arginine | 0.50000 |
| Hydroxyethyl cellulose | 0.43000 |
| Flavor | 0.40000 |
| Methyl paraben | 0.20000 |
| Dibasic potassium phosphate | 0.08000 |
| Potassium chloride | 0.06200 |
| Potassium phosphate monobasic | 0.04300 |
| Calcium chloride dihydrate | 0.01000 |
| Magnesium chloride | 0.00590 |
| Food colorant | 0.00050 |
| Sodium fluoride | 0.00045 |
| TOTAL | 100.00000 |

Formula B

| RAW MATERIAL | WEIGHT % |
|---|---|
| Deionized Water | Q.S. |
| Glycerin | 1.000 |
| 70% Sorbitol | 1.000 |
| Polysorbate 20 | 0.100 |
| Sodium benzoate | 0.010 |
| Calcium chloride | 0.600 |
| Sodium Saccharin | 0.020 |
| Phosphoric acid 85% | 0.080 |
| L-Arginine | 0.600 |
| Flavor | 0.200 |
| Colorants | 0.001 |
| TOTAL | 100.000 |

Formula C

| Material | Weight % |
|---|---|
| Arginine phosphate | 0.5 |
| Sodium benzoate | 0.01 |
| Apple juice | 10 |
| Ascorbic acid | 0.03 |
| Malic acid | 0.15 |
| Flavoring | 0.005 |
| Coloring | 0.004 |
| Calcium carbonate | 0.093 |
| Sodium hydroxide (to adjust pH) | 3.9 |
| Sodium saccharin | 0.020 |
| Demineralized water | q.s. |

1. A beverage comprising an effective amount of
a. arginine in free or salt form, in an amount of at least 0.1% (by weight of free base), and
b. a calcium salt.

2. The beverage of claim 1 further comprising a phosphate ion source.

3. The beverage of claim 1 further comprising a potassium ion source.

4. The beverage of claim 1 further comprising a fluoride ion source.

5. The beverage of claim 1 further comprising a polyol.

6. The beverage of claim 1 further comprising xylitol.

7. The beverage of claim 1 wherein the calcium salt is selected from calcium carbonate, calcium hydroxide, calcium citrate, calcium malate, calcium lactate, calcium chloride, calcium glycerophosphate, calcium formate, and mixtures thereof.

8. The beverage of claim 1 wherein the arginine in free or salt form is arginine bicarbonate.

9. The beverage of claim 1 wherein the arginine in free or salt form is arginine phosphate.

10. The beverage of claim 1 further comprising an organic acid.

11. The beverage of claim 1 which is carbonated.

12. The beverage of claim 1 further comprising fruit juice, fruit extract or fruit concentrate.

13. The beverage of claim 1 further comprising vitamins, minerals, antioxidants, and/or preservatives.

14. A method to:
a. reduce or inhibit formation of dental caries,
b. reduce, repair or inhibit early enamel lesions,
c. reduce or inhibit demineralization and promote remineralization of the teeth,
d. reduce hypersensitivity of the teeth,
e. reduce or inhibit gingivitis,
f. promote healing of sores or cuts in the mouth,
g. reduce levels of acid producing bacteria,
h. increase relative levels of arginolytic bacteria,
i. inhibit microbial biofilm formation in the oral cavity,
j. raise and/or maintain plaque pH at levels of at least pH 5.5 following sugar challenge,
k. reduce plaque accumulation,
l. treat dry mouth,
m. whiten teeth,
n. promote systemic health, including cardiovascular health,
o. reduce erosion of the teeth,
p. immunize the teeth against cariogenic bacteria, and/or
q. clean the teeth and oral cavity;
comprising administering to a subject in need thereof, the beverage of claim 1.

15. The beverage of claim 3, wherein said potassium ion source is selected from potassium nitrate and potassium chloride.

16. The beverage of claim 4, wherein said fluoride ion source is selected from stannous fluoride, sodium fluoride, potassium fluoride, sodium monofluorophosphate, sodium fluorosilicate, ammonium fluorosilicate, amine fluoride, ammonium fluoride, and combinations thereof.
